(19) United States
(12) Patent Application Publication
(10) Pub. No.: US 2013/0303726 A1
(43) Pub. Date: Nov. 14, 2013

(54) METHOD FOR THE PREPARATION OF SURFACTANT PEPTIDES

(71) Applicant: Chiesi Farmaceutici S.p.A., Parma (IT)

(72) Inventors: Andrea MOZZARELLI, Parma (IT); Samanta Raboni, Parma (IT); Barbara Pioselli, Parma (IT)

(21) Appl. No.: 13/862,590

(22) Filed: Apr. 15, 2013

(30) Foreign Application Priority Data
Apr. 17, 2012 (EP) 12002678.6

(51) Int. Cl. C07K 14/785; C12P 21/06

(52) U.S. Cl. CPC C07K 14/785; C12P 21/06
USPC 530/300; 435/320.1; 435/68.1

(57) ABSTRACT

Surfactant-protein C peptides (SP-C peptides) may be prepared by the heterologous expression of a fusion protein of an SP-C peptide and a maltose binding protein.

### CROSS REFERENCES TO RELATED APPLICATIONS

This application claims priority to European Patent Application No. 12002678.6 filed on Apr. 17, 2012, which is incorporated herein by reference in its entirety.

### BACKGROUND OF THE INVENTION

1. Field of the Invention

The present invention relates to methods based on recombinant-DNA technology for the preparation of surfactant-protein C peptides (SP-C peptides). The present invention also relates to genetic constructs, vectors, and host cells for use in such a method.

2. Discussion of the Background

Pulmonary surfactant reduces surface tension at the air-liquid interface of the alveolar lining, preventing the lungs from collapsing at the end of expiration. Surfactant deficiency is a disorder in premature infants and causes respiratory distress syndrome (RDS), which can be effectively treated with natural surfactants extracted from animal lungs (see Fujiwara, T. and Robertson B. (1992) In: Robertson, B., van Golde, L. M. G. and Batenburg, B. (eds) Pulmonary Surfactant: From Molecular Biology to Clinical Practice Amsterdam, Elsevier, pp. 561-592).

The main constituents of these surfactant preparations are phospholipids such as 1,2-dipalmitoyl-sn-glycero-3-phosphocholine (DPPC), phosphatidylglycerol (PG), and the hydrophobic surfactant proteins B and C(SP-B and SP-C). The proteins SP-B and SP-C constitute only about 1-2% of the surfactant mass, but are still able to exercise dramatic improvements on surface activity, compared to pure lipid preparations (see Curstedt, T. et al. (1987) Eur. J. Biochem. 168, 255-262;).

SP-C is a lipoprotein composed of 35 amino acid residues with an alpha-helical domain between residues 9-34 (see Johansson, J. et al. (1994) Biochemistry 33, 6015-6023). The helix is composed mostly of valyl-residues and is embedded in a lipid bilayer and oriented in parallel with the lipid acyl chains (see Vandenbussche, et al. (1992) Eur. J. Biochem. 203, 201-209). Two palmitoyl groups are covalently linked to cysteine residues in positions 5 and 6 in the N-terminal part of the peptide (see Curstedt, T. et al. (1990) Proc. Natl. Acad. Sci. U.S.A. 87, 2985-2989).

The two conserved positively charged residues, arginine and lysine, at positions 11 and 12, possibly interact with the negatively charged head groups of the lipid membrane, thus increasing its rigidity. The rigidity of the lipid-peptide interaction may be decreased towards the C-terminal end, since it contains small or hydrophobic residues only, making this part potentially more mobile in a phospholipid bilayer.

Since surfactant preparations obtained from animal tissue may present some drawbacks, like their availability in limited amounts and the possibilities that they contain infectious agents and induce immunological reactions, attempts have been made to create artificial surfactants usually constituted of synthetic lipids and synthetic analogues of the SP-C and/or SP-B proteins. However, as far as the synthetic SP-C protein is concerned, previous work has demonstrated that it may not fold like the native peptide into an alpha-helical conformation necessary for optimal surface activity (see Johansson, J. et al. (1995) Biochem. J. 307, 535-541), and therefore might not interact properly with the surfactant lipids.

To circumvent this problem, several attempts have been made to modify the sequence, for instance by replacing all helical Val residues in native SP-C with Leu, which strongly favors alpha-helical conformation. The corresponding transmembranous analogue, SP-C(Leu) showed good spreading at an air-liquid interface when combined with appropriate phospholipids mixtures.

WO 2008/044109 discloses the peptide analog of the SP-C protein, quoted as SP-C33(Leu), having the following one-code amino acid sequence

### IPSSPVHLKRLKLLLLLLLLILLLILGALLLGL (SEQ ID NO:1).

This peptide may be prepared by synthetic methods. Conventional synthetic methods are described, for instance, in Schroeder et al., ‘The peptides’, vol. 1, Academic Press, 1965; Bodanszky et al., ‘Peptide synthesis’, Interscience Publisher, 1996; and Baramy & Merrifield, ‘The peptides; Analysis, Synthesis, Biology’, vol. 2, chapter 1, Academic Press, 1980, all of which are incorporated herein by reference in their entireties. Said techniques include peptide synthesis in solid phase, in solution, organic chemistry synthetic methods, or any combination thereof. The production by conventional techniques of synthetic peptides such as the peptide SP-C33(Leu) is affected by their high hydrophobic character which limits their water solubility.

Thus, there remains a need for improved methods of preparing surfactant-protein C peptides (SP-C peptides)

### SUMMARY OF THE INVENTION

Accordingly, it is one object of the present invention to provide novel recombinant methods for the biosynthesis of an SP-C peptide, which overcome the drawbacks associated with conventional techniques and enable the attainment of highly pure product with satisfactory yields.

This and other objects, which will become apparent during the following detailed description, have been achieved by the inventors' discovery of methods based on the expression of a fusion protein in which the SP-C peptide is fused to the maltose-binding protein (MBP) by interposition of a linker carrying a protease cleavage site.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is an analysis of the MALDI spectrum and reveals that SP-C33(Leu) is present within the solution upon digestion. The calculated monoisotopic mass is 3594.52 Da which is within 22 ppm with respect to the theoretical monoisotopic mass calculated from the amino acid sequence.

FIG. 2 shows the presence of the MBP-linker (MWₐᵥₑ˜43206 Da) as determined by a lower resolution MALDI-MS approach. No detectable amounts of MBP-linker-SP-C33(Leu) were observed, suggesting that the applied proteolytic reaction conditions result in a quantitative process.

FIG. 3 is a preliminary evaluation of impurities was also performed by MALDI-MS and HPLC-ESIMS in between two batches, labelled 1 and 2, obtained independently. They showed a comparable level of purity. Quantification of the product SP-C33(Leu) by LC-MS was performed against a SP-C33(Leu) reference standard. The two batches obtained independently were measured and the yield in protein was determined (see Table 1).

### DETAILED DESCRIPTION OF THE PREFERRED EMBODIMENTS

By the term “SP-C peptide” it is meant peptides structurally analogous to the native surfactant protein SP-C, including peptides having an amino acid sequence in which, compared to the native protein, one or ore amino acids are substituted and/or missing so long as said peptides, in a mixture with a lipid carrier, show a similar pulmonary surfactant activity. Suitable SP-C peptides also include those described in U.S. Pat. No. 5,874,406, which is incorporated herein by reference in its entirety.

In one embodiment, the present invention provides an expression cassette containing a polynucleotide sequence encoding an SP-C peptide, an MBP protein and a linker peptide located therebetween carrying a protease cleavage site, said encoding polynucleotide sequence being operatively linked to a promoter sequence suitable for the expression in a prokaryotic cell.

In one embodiment, the SP-C peptide is SP-C33(Leu) (SEQ ID NO:1) and the encoding sequence is SEQ ID NO:3. In another embodiment, MBP is identified by SEQ ID NO:2 and its encoding sequence is SEQ ID NO:4. Compared to the wt MBP—i.e. the one naturally produced by E. coli—the maltose binding protein SEQ ID NO:2 presents mutations that confer improved affinity for amylose and better folding of the SPC peptide. An improved amylose-affinity is important for the purification of the fusion protein produced by bacterial cells, as discussed below.

The MBP- and SP-C-encoding sequences are preferably located at the N- and C-terminus of the expression cassette, respectively. It was found that this facilitates the correct folding of the fused SP-C peptide.

In yet another embodiment, the linker peptide is 10 to 50, preferably 20 to 40 amino acid residues long and contains a proteolysis site recognized by an enterokinase. The latter is a specific serine protease that cleaves after lysine at a specific cleavage site. In addition the encoding sequence of the linker may contain one or more endonuclease-restriction sites for suitable cloning and processing of the genetic construct. In a preferred embodiment, the linker peptide and its encoding sequence are identified by SEQ ID NOs:5 and 6, respectively.

Any promoter suitable for regulating the expression of heterologous proteins in a prokaryotic cell can be used according to the invention. Preferably an inducible promoter is used and particularly the tac promoter, which is activated by isopropyl beta-D-1-thiogalactopyranoside (IPTG).

The expression cassette may further include components that modulate the expression of the recombinant protein, such as transcription enhancers, terminators, initiators and other genetic control elements or elements conferring binding affinity or antigenicity to the recombinant protein.

In a further embodiment the invention relates to an expression vector containing the expression cassette described above. Preferably the vector is a plasmid and more preferably a pBR32-based plasmid, which may additionally contain selection markers such as antibiotic resistance encoding sequences, secretion signals directing the recombinant protein to a secretory pathway and suitable restriction sites to allow insertion of the heterologous sequences.

In a yet further embodiment, the invention provides a method for the preparation of an SP-C peptide which comprises the following steps:

i) providing a vector carrying an expression cassette, wherein the expression cassette contains a polynucleotide sequence encoding a fusion protein consisting of an SP-C peptide, MBP, and a linker peptide there between carrying a protease cleavage site, as defined above;

ii) introducing said vector into a prokaryotic cell and maintaining the cell under conditions permissive for the expression of the fusion protein;

iii) purification of the fusion protein;

iv) cleavage of the fusion protein with a suitable protease and isolation of the SPC protein.

Bacterial cells and particularly E. coli cells are conveniently used for the expression of the SPC peptide. E. coli strains that contain genetic mutations phenotypically selected for conferring tolerance to toxic proteins are particularly preferred.

After expression of the fusion protein, the cells are disrupted and the cellular lysate centrifuged to separate the cell fractions including the fusion protein. The purification of the fusion protein is preferably carried out by means of affinity chromatography using a MBP ligand-bound resin. Preferably, the crude cell extract is loaded over a column containing an agarose resin derivatized with amylose and eluted with a buffered solution containing a suitable amount of maltose to remove the fusion protein from the resin.

The final cleavage of the fusion protein liberates the SP-C peptide which is then isolated using conventional techniques. The enterokinase protease is preferably used to cleave the fusion protein in the linker region, where suitable protease cleavage sites are present.

Unlike other protein tags tested in similar recombinant systems, MBP proved particularly effective for the expression and subsequent purification of the SP-C peptide fused thereto.

Other features of the invention will become apparent in the course of the following descriptions of exemplary embodiments which are given for illustration of the invention and are not intended to be limiting thereof.

### Examples

### Cloning Strategy for the MBP Fusion Protein.

SP-C33(Leu) is expressed in bacteria in the form of a fusion protein with the maltose binding protein (MBP). The expression vector pMALc5e (New England Biolabs) codes for MBP and provides for a (5′)AvaI and a (3′)BamHI cleavage site for the in-frame cloning of the DNA fragment coding for SpC33Leu. The expression vector contains an ampicillin resistance gene and is a derivative of pBR322. MBP and SpC33Leu are connected together via a peptide linker that contains a proteolysis site recognized by enterokinase, a specific serine protease that cleaves after lysine at a specific cleavage site (Asp-Asp-Asp-Asp-Lys↓). The commercial MBP-linker sequence consists of the maltose binding protein from E. coli preceded by methionine and with the final 4 amino acids replaced by 21 residues encoded by the polylinker of pMAL-c5e plus a C-terminal glycine.

The original commercial polylinker has been modified to include several new endonuclease restriction sites (SEQ ID NO:5 and 6).

The SP-C33(Leu) nucleotide sequence is synthesized and cloned in a suitable shuttle vector by GeneArt® Gene Synthesis service. The nucleotide sequence is optimized by GeneArt® Gene Synthesis service according to codon usage frequency in E. coli. The codon-optimization changes only the nucleic acid sequence and not the encoded amino acid sequence. Gene design and optimization strategy use the proprietary GeneOptimizer® software (see WO-A-04/059556 and WO-A-06/013103, which are incorporated herein by reference in their entireties) [Raab D., Graf M., Notka F., Schödl T. and Wagner R. “The GeneOptimizer Algorithm: using a sliding window approach to cope with the vast sequence space in multi-parameter DNA sequence optimization” Syst Synth Biol. 2010; 4: 215-225; Maertens B., Spriestersbach A., von Groll U., Roth U., Kubicek J., Gerrits M., Graf M., Liss M., Daubert D., Wagner R., and Scha\ddot{}fer F. “Gene optimization mechanisms: A multi-gene study reveals a high success rate of full-length human proteins expressed in Escherichia coli” Protein Science 2010; 19: 1312-1326, which are incorporated herein by reference in their entireties]. Optimization parameters include codon usage, DNA motifs such as ribosomal entry sites, GC content and avoidance of (inverted) repeats. The sequence codes for i) an AvaI-specific 5′, ii) a BamHI-specific 3′ end for subsequent subcloning, iii) the enterokinase cleavage site preceding SpC33Leu sequence, and provides a TAA stop codon to terminate ribosomal translation. AvaI and BamHI are restriction endonucleases. AvaI recognizes the double-stranded nucleotide sequence CYCGRG (where Y=T/C, and R=A/G) and cleaves after C-1. BamHI recognizes the sequence GGATCC, and cleaves after G-1. Both enzymes produce a cohesive end. The optimized genes are then assembled by synthetic oligonucleotides (de novo gene synthesis), cloned into pMA-T vector using SfiI and SfiI cloning sites. The final construct is sequence verified.

Preparation of the plasmid for expressing SpC33Leu, DNA amplification, plasmid isolation, purification, and transformation in electrocompetent E. coli cells, were carried out using conventional protocols of recombinant DNA technology that are discussed generally in Sambrook and Russel, Molecular Cloning: A Laboratory Manual, CSHL Press 2001, which is incorporated herein by reference in its entirety.

For subcloning, SpC33Leu sequence is digested with AvaI and BamHI to produce protruding single-stranded ends. Incorporation into the vector takes place after AvaI/BamHI digestion of the vector, dephosphorylation, purification of the required vector DNA fragment by agarose gel electrophoresis and hybridization of SpC33Leu fragment and the vector fragment via the cohesive ends. Subsequently the two fragments are covalently linked by ligation using T4 DNA ligase (New England Biolab), followed by transformation into bacteria host cells. Selection of plasmid-harboring cells was carried out by plating on LB agar plates with ampicillin. Plasmid DNA is isolated from the resulting Amp-resistant colonies and is analyzed with suitable restriction enzymes. Clones with the expected DNA restriction fragment pattern are selected. Complete sequencing by BMR Genomics (University of Padua) of the plasmid sequence confirms the correct insertion of the SpC33Leu sequence.

### Producer Strain.

The producer strains E. coli C41(DE3) and C43(DE3), which are used for the expression of SP-C33(leu) are derived from E. coli BL21(DE3) and can be purchased from Lucigen. These strains are reported to be effective in over-expressing toxic and membrane proteins of viral, eubacterial, archaeal, plant, yeast, drosophila or mammalian origin, since these strains have at least one uncharacterized mutation, which prevents cell death associated with expression of many recombinant toxic proteins.

### Protein Expression.

The recombinant plasmid permits expression of the fusion protein MBP-SpC33Leu under control of the tac promoter. The recombinant fusion protein is produced in soluble form in the host cells after induction with Isopropyl β-D-1-thiogalactopyranoside (IPTG).

1 ml preculture or starter culture (Luria-Bertani medium: 10 g/l tryptone, 5 g/l yeast extract, and 10 g/l NaCl in the presence of 10 mM glucose) is inoculated with a glycerol culture seeded on a LB plate and incubated under strong ampicillin selection pressure at 37° C. with shaking overnight. The culture is used to inoculate 5 mL of culture. Growth of bacteria is continued until it reaches an optical density of about 0.6 at 600 nm. Culture is then induced by adding IPTG. After induction, growth is continued for 4-5 hours until the cells are harvested by centrifugation at 4° C. The moist biomass is resuspended in 20 mM NH₄HCO₃ buffer, pH 7.5. Cell suspension is disrupted by sonication in ice. After lysis of the bacteria, the expression mixture is checked on a 12% Laemmli reducing SDS-PAGE gel to evaluate the total protein content. Identity of the new band as the fusion protein is confirmed by immunoblotting using rabbit anti-MBP antiserum (NEB). Blotting on a nitrocellulose membrane was performed in a semi-dry apparatus at 10V for 40 minutes. Tris Buffered Saline, pH 8.0, with 3% nonfat milk is used as blocking buffer. Primary antibody is diluted 1:10,000 in blocking buffer and incubated for 1 hour at room temperature. Anti-rabbit IgG-peroxidase conjugate was used as the secondary antibody in conjunction with horseradish peroxidase substrate, 3,3′,5,5′-tetramethylbenzidine (TMB), for detection (all immunoblotting reagents were purchased from Sigma Aldrich). After induction a new predominant band of the correct molecular weight for the fusion protein corresponding to the combination of MBP and SpC33Leu fragment is detected both by SDS-PAGE and immunoblotting.

### Purification of the Fusion Protein.

Isolation of the fusion protein from the expression mixture is facilitated by MBP affinity chromatography. The crude cell extract is loaded over a column containing an agarose resin derivatized with amylose (pMAL protein fusion and purification system, New England Biolabs). The fusion protein binds to the column because of MBP affinity for amylose and is eluted with 20 mM NH₄HCO₃ buffer, pH 7.5, containing 10 mM maltose. The eluate containing the fusion protein of interest is analyzed on a 12% Laemmli reducing SDS-PAGE gel and identified by immunoblot using anti-MBP serum.

The yield of MBP-SpC33Leu fusion protein is 50-80 mg from a liter of culture. Subsequent cleavage with enterokinase to separate MBP and SP-C33(Leu) takes place between Lys-393 and Ile-394 in MBP-SpC33Leu. Ile-394 corresponds to the first amino acid of SP-C33(Leu) peptide.

### Cleavage of the Fusion Proteins.

To cleave the fusion protein at the Lys-393 connection amino acid, 0.02 units of enterokinase per mg of fusion protein are added to the solution. 1% (v/w) Triton X-100 was added to the cleavage solution in order to maintain the solubility of the cleaved digestion hydrophobic product SpC33Leu. The solution was left to stand for 16 hours with gentle stirring at room temperature.

### Mass Spectrometric Characterization of the Protein Product SpC33Leu.

Two different procedures were applied in order to characterize the digestion product of MBP-SpC33Leu fusion protein. A MALDI-MS high resolution approach was aimed at determining the monoisotopic molecular weight of the product and evaluating the expression of the correct sequence and the correct cleavage of the fusion protein by enterokinase. HPLC-ESMS was chosen as method of choice to preliminarily evaluate the impurities profiling of the recombinant product with respect to a reference standard and to evaluate the reproducibility of the batches.

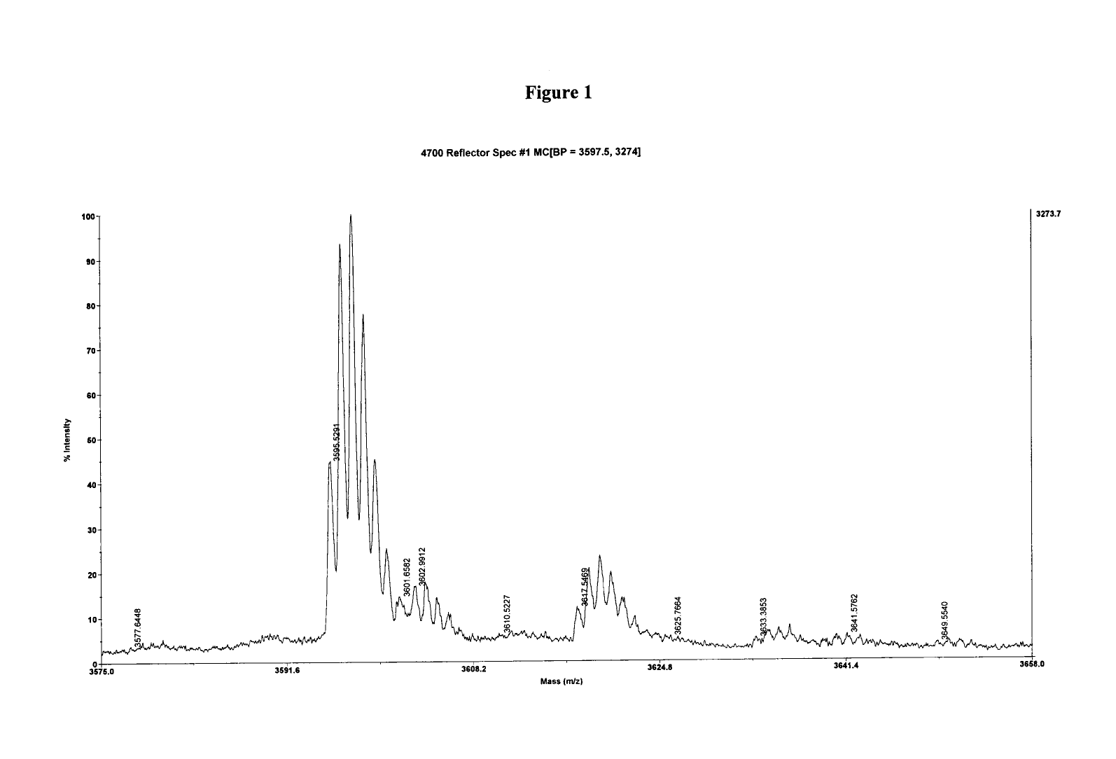

FIG. 1 shows an analysis of the MALDI spectrum and reveals that SP-C33(Leu) is present within the solution upon digestion. The calculated monoisotopic mass is 3594.52 Da which is within 22 ppm with respect to the theoretical monoisotopic mass calculated from the amino acid sequence.

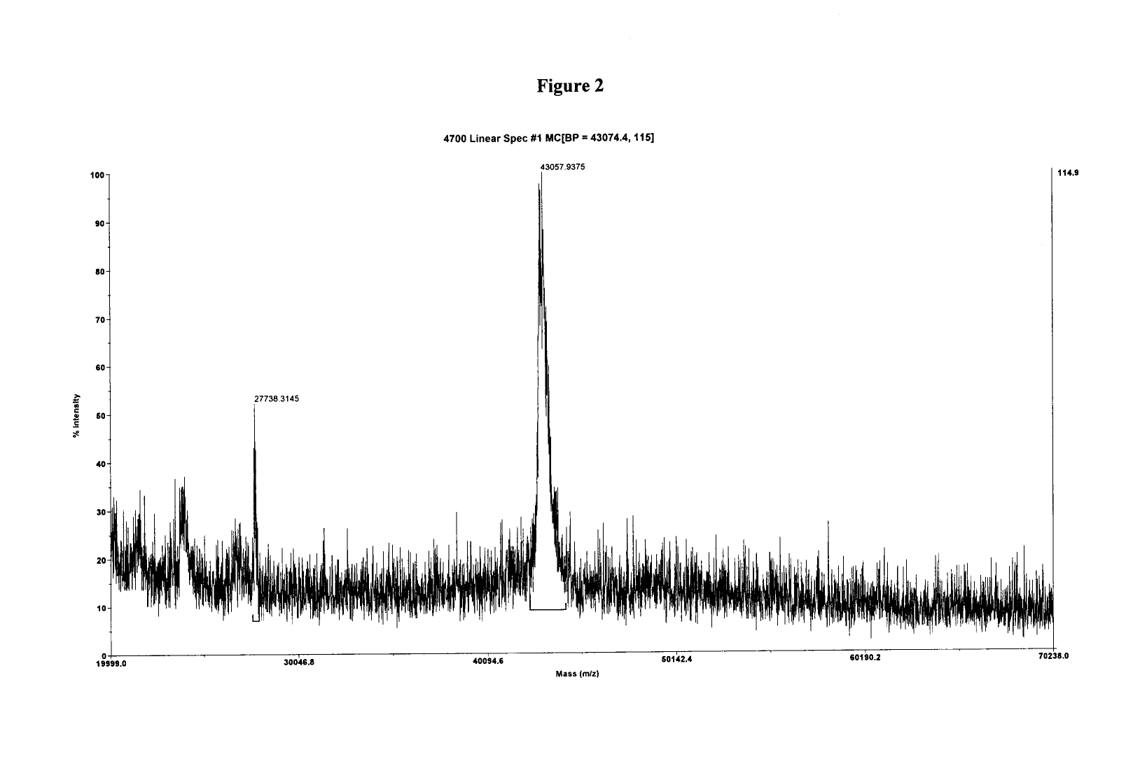

FIG. 2 shows the presence of the MBP-linker (MWₐᵥₑ˜43206 Da) as determined by a lower resolution MALDI-MS approach. No detectable amounts of MBP-linker-SP-C33(Leu) were observed, suggesting that the applied proteolytic reaction conditions result in a quantitative process.

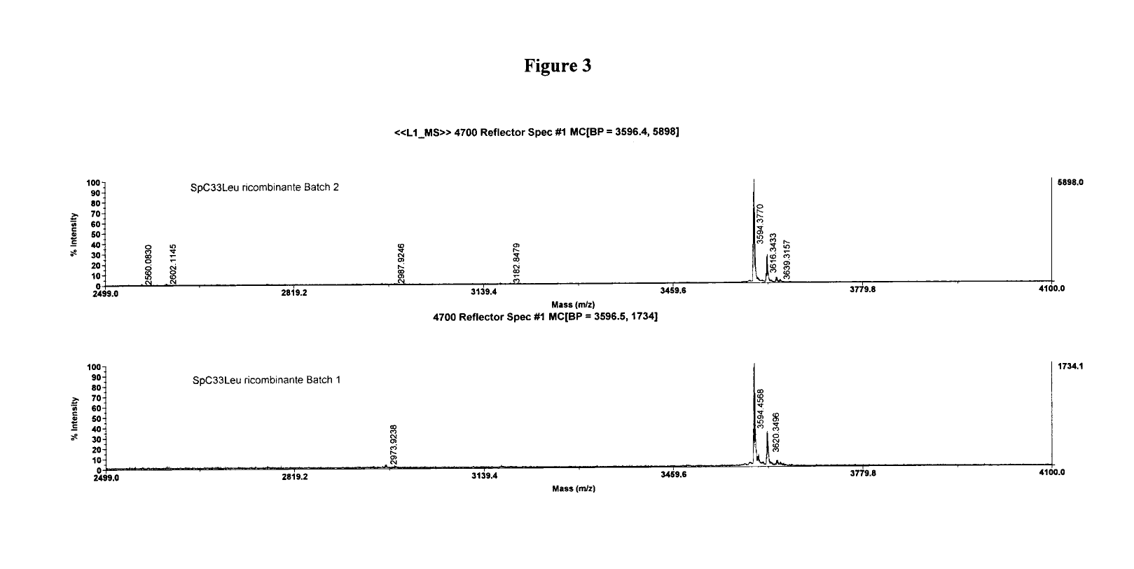

FIG. 3 is a preliminary evaluation of impurities was also performed by MALDI-MS and HPLC-ESIMS in between two batches, labelled 1 and 2, obtained independently. They showed a comparable level of purity. Quantification of the product SP-C33(Leu) by LC-MS was performed against a SP-C33(Leu) reference standard. The two batches obtained independently were measured and the yield in protein was determined (see Table 1).

TABLE 1

| | Batch average calculated | Yield (mg) |
|---|---|---|
| Batch # | concentration (mg/ml) | per liter of culture |
| SP-C33(Leu) | 0.048 | 8.8 |
| Batch 2 | | |
| SP-C33(Leu) | 0.048 | 8.7 |
| Batch 1 | | |

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An expression cassette, comprising:
(a) a polynucleotide sequence encoding an SP-C peptide;
(b) a polynucleotide sequence encoding an MBP protein; and
(c) a polynucleotide sequence encoding a linker peptide,
wherein said a polynucleotide sequence encoding a linker peptide encodes a protease cleavage site and is located between said polynucleotide sequence encoding an SP-C peptide and said polynucleotide sequence encoding an MBP protein, and
said encoding polynucleotide sequence being operatively linked to a promoter sequence suitable for the expression in a prokaryotic cell.

2. An expression cassette according to claim 1, wherein said SP-C peptide is SP-C33(Leu) and has the sequence shown in SEQ ID NO:1.

3. An expression cassette according to claim 1, wherein the MBP protein has the sequence shown in SEQ ID NO:2.

4. An expression cassette according to claim 1, wherein said polynucleotide sequence encoding an SPC peptide has the sequence shown in SEQ ID NO:3.

5. An expression cassette according to claim 1, wherein said polynucleotide sequence encoding an MBP protein has the sequence shown in SEQ ID NO:4.

6. An expression cassette according to claim 1, wherein said linker peptide has the amino acid sequence shown in SEQ ID NO:5.

7. An expression cassette according to claim 1, wherein said polynucleotide sequence encoding a linker peptide has the sequence shown in SEQ ID NO:5.

8. An expression cassette according to claim 1, wherein said polynucleotide sequence encoding an MBP protein is located at the N-terminus.

9. An expression cassette according to claim 1, wherein said polynucleotide sequence encoding an SP-C peptide is located at the C-terminus.

10. An expression cassette according to claim 1, wherein said polynucleotide sequence encoding an MBP protein is located at the N-terminus and said polynucleotide sequence encoding an SP-C peptide is located at the C-terminus.

11. An expression cassette according to claim 1, wherein said promoter is an inducible tac promoter.

12. An expression vector, containing an expression cassette according to claim 1.

13. An expression vector according to claim 12, which is a plasmid.

14. An expression vector according to claim 13, which is a pBR32-based plasmid.

15. A method for preparing an SP-C peptide, which comprises:
(i) providing a vector carrying an expression cassette according to claim 1;
(ii) introducing said vector into a prokaryotic cell and maintaining said cell under conditions permissive for the expression of the fusion protein encoded by said casette;
(iii) purifying said fusion protein;
(iv) cleaving said fusion protein with a suitable protease; and
(v) isolating the SP-C peptide.

16. A method according to claim 15, wherein said prokaryotic cell is an E. coli bacterial cell.

17. A method according to claim 15, wherein said purifying of said fusion protein is carried out by affinity chromatography using a MBP ligand-bound resin.

18. A method according to claim 15, wherein an enterokinase protease is used for said cleaving said fusion protein.

19. An SP-C peptide, which is prepared by a method according to claim 15.

20. A pulmonary surfactant composition, which comprises an SP-C according to claim 19.


SEQUENCE LISTING (6 sequences)

SEQ ID NO: 1 - protein, 33 aa, Artificial Sequence
  synthetic SP-C peptide
IPSSPVHLKRLKLLLLLLLLILLLILGALLLGL

SEQ ID NO: 2 - protein, 368 aa, Artificial Sequence
  mutated form of MBP produced by E. coli
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTN

SEQ ID NO: 3 - DNA, 99 bp, Artificial Sequence
  SP-C encoding sequence
attccgagcagtccggttcatctgaaacgtctgaaactgttattactgctgttactgctgattctgctgttaattctgggtgcactgctgctgggtctg

SEQ ID NO: 4 - DNA, 1106 bp, Artificial Sequence
  mutated MBP-encoding sequence
ttatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaat

SEQ ID NO: 5 - protein, 25 aa, Artificial Sequence
  synthetic linker peptide
SSSNNNNNNNNNNLGPDLGTDDDDK

SEQ ID NO: 6 - DNA, 75 bp, Artificial Sequence
  linker-peptide encoding sequence
tcgagctcgaacaacaacaacaataacaataacaacaacctcgggccagatctgggtaccgatgatgatgataaa